(19) United States
(12) Patent Application Publication
(10) Pub. No.: US 2018/0296302 A1
(43) Pub. Date: Oct. 18, 2018

(54) MARKER SYSTEM FOR ENCODED INFORMATION AT BIOPSY SITES

(71) Applicant: David GRUEN, Weston, CT (US)

(72) Inventor: David GRUEN, Weston, CT (US)

(21) Appl. No.: 15/956,439

(22) Filed: Apr. 18, 2018

Related U.S. Application Data
(60) Provisional application No. 62/486,564, filed on Apr. 18, 2017.

(51) Int. Cl. A61B 90/00; A61B 10/02

(52) U.S. Cl. CPC A61B 90/39; A61B 10/0233; A61B 2090/3925; A61B 2090/3908; A61B 2090/3987; A61B 2090/3954

(57) ABSTRACT

Methods and apparatus for marking a biopsy site call for deposition of a marker that includes an appearance that is correlated to the surgical modality and provides related information to future practitioners evaluating the biopsy site.

### CROSS REFERENCE TO RELATED APPLICATIONS

This application is filed under 35 U.S.C. § 111(a) and further, under 35 U.S.C. § 119(e), claims the benefit of U.S. Provisional Patent Application Ser. No. 62/486,564, filed Apr. 18, 2017 and entitled “Marker System for Encoded Information at Biopsy Sites,” the entire disclosure of which is incorporated herein by reference for any purpose whatsoever.

### BACKGROUND OF THE INVENTION

### 1. Field of the Invention

The invention disclosed herein relates to biopsy marker systems, and in particular to methods and apparatus that enable accurate identification of biopsy sites.

### 2. Description of the Related Art

Healthcare systems worldwide involve substantial costs. Any commonly occurring disease presents both financial and emotional burdens to those involved. Consider, for example, the impacts of performing biopsy screenings for breast and lymph node diagnoses.

In the United States, approximately 1.7 million breast biopsies are performed on an annual basis. Worldwide, approximately 5 million breast biopsies are performed on an annual basis.

In order to facilitate the ongoing process of diagnosis and therapy, biopsy markers have been developed. Generally, a biopsy marker is placed after a biopsy to help locate the site for future reference. As a substantial portion, if not all of a lesion may be removed during the biopsy procedure, locating the site could potentially be difficult after the biopsy. Accordingly, a small marker (that is typically less than about 3 mm in size) is deposited by a practitioner. The marker is readily identifiable with diagnostic imaging, thus enabling future therapy to be precisely targeted. At least, that has been the design.

That is, in simple situations use of a biopsy marker is uncomplicated. However, some patients have a history and may be carrying biopsy markers that were placed sometime in the past, and some patients have multiple markers in their breast. During imaging, all of the biopsy markers will appear. It may be hard, if not impossible, to distinguish one marker from another. That is, while the various shapes and sizes and types of markers differ, and one marker may be physically different than another, it may be impossible to tell which marker is presently relevant.

This is a pervasive issue. Markers are deployed in essentially all image-guided breast biopsies. As current marker options are neither site nor modality specific, presently available markers are often confusing to physicians and a potential source of error.

In one example, a fifty five year old female was evaluated. The patient presented with four prior biopsies of the right breast and two of the left breast. A fifth biopsy on the right breast showed breast cancer. The patient was then referred to a breast surgeon to remove the cancer. The problem was properly identifying which marker indicated the cancer.

What are needed are methods and apparatus to allow precise marking of biopsy sites. Preferably, the methods and apparatus provide practitioners with meaningful information while remaining relatively simple and easy to use.

### SUMMARY OF THE INVENTION

In one embodiment, a surgical marker is provided. The surgical marker includes a biocompatible material encoded with information of medical significance, the surgical marker exhibiting a geometry suited for being disposed at a location of interest within a patient and remaining in-situ to signify the information.

A shape of the surgical marker may include at least one alphanumeric character and the at least one alphanumeric character may derived from at least one of the English alphabet and Arabic numbers. The encoded information may indicate at least one of a surgical modality and a sequence within a series of surgeries. The surgical marker may include at least one retention feature, the at least one retention feature may include a hook, a barb, a protuberance, a point, a jagged edge, a roughened surface, a porous surface and/or any other type of surface deemed appropriate for assisting with limiting the mobility of the marker. The biocompatible material may include at least one of: metal, stainless steel, a cobalt alloy, a titanium alloy; a ceramic material, aluminum oxide, zirconia, calcium phosphate and a polymeric material. The biocompatible material may include shape-memory material. The surgical marker may be configured for delivery to the location of interest by use of a cannula. The surgical marker may further include at least one contrast enhancing agent incorporated therewith. The surgical marker may further include at least one antibiotic agent incorporated therewith.

In another embodiment, a method for marking a biopsy site with a surgical marker is provided. The method includes selecting a surgical marker including a biocompatible material encoded with information of medical significance, the surgical marker exhibiting a geometry suited for being disposed at a location of interest within a patient and remaining in-situ to signify the information; and, implanting the surgical marker at a surgical site.

The selecting may include choosing the surgical marker according to the encoded information in correlation with at least one of a modality and a sequence in a series of surgeries. The selecting may further include one of obtaining an instrument with the surgical marker loaded therein and loading an instrument with the surgical marker. The implanting may include one of manually disposing the surgical marker at the surgical site and operating an instrument for disposing the surgical marker at the surgical site.

In a further embodiment, a method for fabrication of a surgical marker for marking a surgical site is provided. The method includes selecting at least one biocompatible material for forming the surgical marker; and, forming the surgical marker into a geometry suited for being disposed at a location of interest within a patient and remaining in-situ to signify encoded information of medical significance. The method may further include at least one of molding, machining, casting, assembling, melting, solidifying, polishing the surgical marker. The method may further include at least one of packaging and sterilizing the surgical marker. The selecting may include choosing the biocompatible material from at least one of metal, stainless steel, a cobalt alloy, a titanium alloy; a ceramic material, aluminum oxide, zirconia, calcium phosphate, a polymeric material and shape-memory material.

### BRIEF DESCRIPTION OF THE DRAWINGS

The features and advantages of the invention are apparent from the following description taken in conjunction with the accompanying drawings in which:

FIG. 1 is an illustration of prior art surgical markers;

FIG. 2 is an illustration of tissue containing a distribution of prior art surgical markers;

FIG. 3 is an illustration of four series of surgical markers according to the teachings herein;

FIG. 4 is an illustration of one of the surgical markers of FIG. 3;

FIGS. 5A and 5B, collectively referred to herein as FIG. 5, depict aspects of a trocar for implantation of surgical markers according to the teachings herein;

FIG. 6 is a flow chart depicting an exemplary method for manufacturing surgical markers according to the teachings herein; and

FIG. 7 is a flow chart depicting an exemplary method for implanting a surgical marker according to the teachings herein.

### DETAILED DESCRIPTION OF THE INVENTION

Disclosed herein are methods and apparatus for marking a biopsy site. In general, the techniques call for deposition of a marker (e.g., a metallic marker) that includes an appearance that is correlated to the specifics of that unique biopsy procedure and provides related information to future practitioners evaluating the biopsy site. Prior to discussing the methods and apparatus for marking a biopsy site in detail, some context is provided.

As discussed herein, the terms “marker” and “clip” are interchangeable. These terms generally refer to devices configured for being deposited at a surgical site in order to provide future referencing for further diagnostics or therapy. Biopsy markers are often fabricated from biologically inert materials such as titanium.

As discussed herein, the term “modality” generally refers to types of procedures that may make use of a marker or clip. Examples of modalities used include, without limitation: magnetic resonance imaging (MRI); ultrasound; mammography (stereotactic) guided biopsy; and lymph node biopsy. As discussed herein, the term “stereotactic” or “stereotaxy” generally refers to a minimally invasive form of surgical intervention which makes use of a three-dimensional coordinate system to locate small targets inside the body and to perform on them some action such as ablation, biopsy, lesion, injection, stimulation, implantation and radiosurgery (SRS). Generally, the modality elected is with reference to a particular biopsy procedure, but this is not limiting of the teachings herein.

As discussed herein, the term “biopsy site” generally refers to a location of interest (having a given volume) within the tissue of a patient where marking is to be undertaken. The biopsy site may be identified for further clinical analysis, therapy or any other reason deemed appropriate. The clinical analysis may be for evaluation of potentially cancerous tissue or for other reasons.

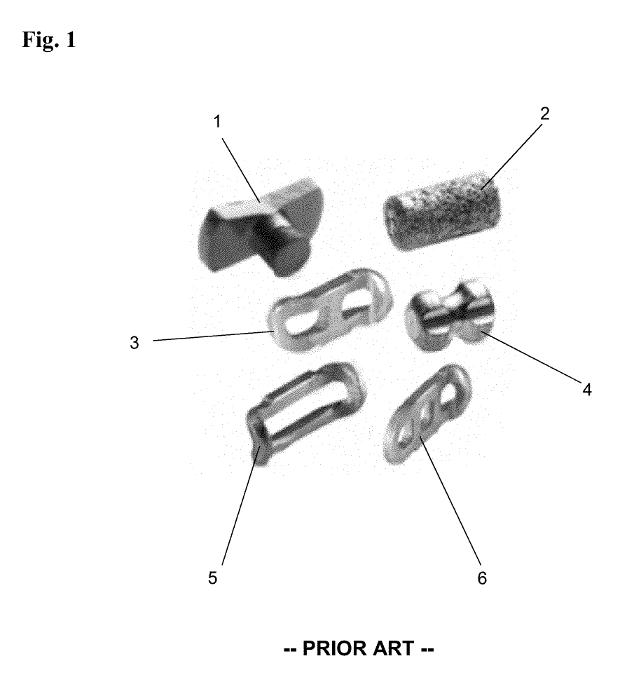

FIG. 1 depicts various prior art surgical markers 1, 2, 3, 4, 5 and 6. Each of the prior art surgical markers 1, 2, 3, 4, 5 and 6 exhibit a particular shape. The appearance of each of the prior art surgical markers 1, 2, 3, 4, 5 and 6 is indicative of a manufacturer. As may be seen, the prior art surgical markers 1, 2, 3, 4, 5 and 6 do not, on an intuitive level, convey any information whatsoever. Thus, while the prior art surgical markers 1, 2, 3, 4, 5 and 6 are readily apparent through imaging techniques, the prior art surgical markers 1, 2, 3, 4, 5 and 6 do not, in of themselves, convey any additional information about the specifics of the unique biopsy procedure.

Accordingly, the methods and apparatus provided herein include a series of markers useful for marking a biopsy site within a patient. Each of the markers include coding that may readily signify information. For example, each of the markers may include an alphanumeric appearance that signifies aspects of medical significance. More specifically, and as an example, each of the markers may include one of an M, U, S, or N. By use of the M, U, S, or N a subsequent observer may intuitively associate the marker with a given modality (where M signifies magnetic resonance imaging (MRI); where U signifies ultrasound; where S signifies stereotactic (mammographic) guided biopsy; and where N signifies lymph node biopsy). Similarly, incorporation of a numeric appearance within the marker may serve to indicate a sequence within a series. That is, 1 signifies a first biopsy procedure performed with a given modality; 2 signifies a second biopsy procedure with the modality, and so on.

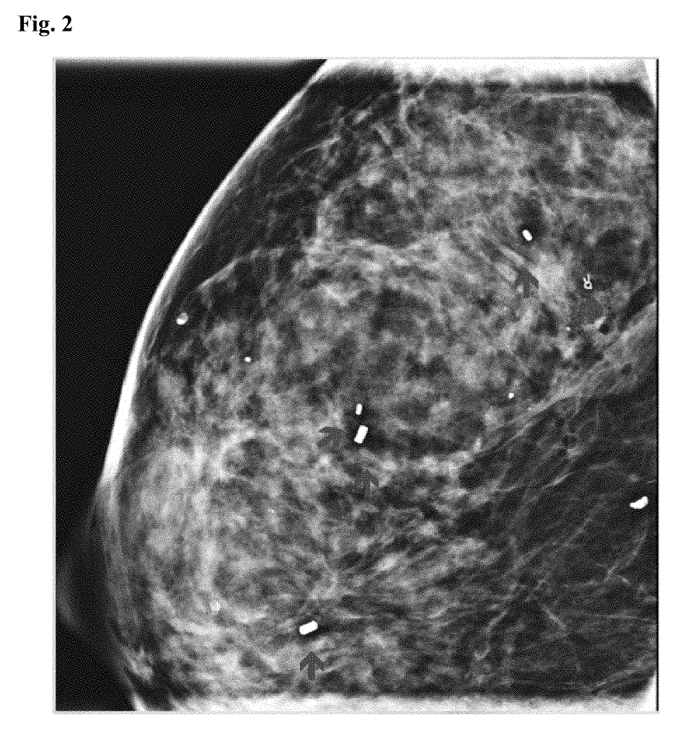

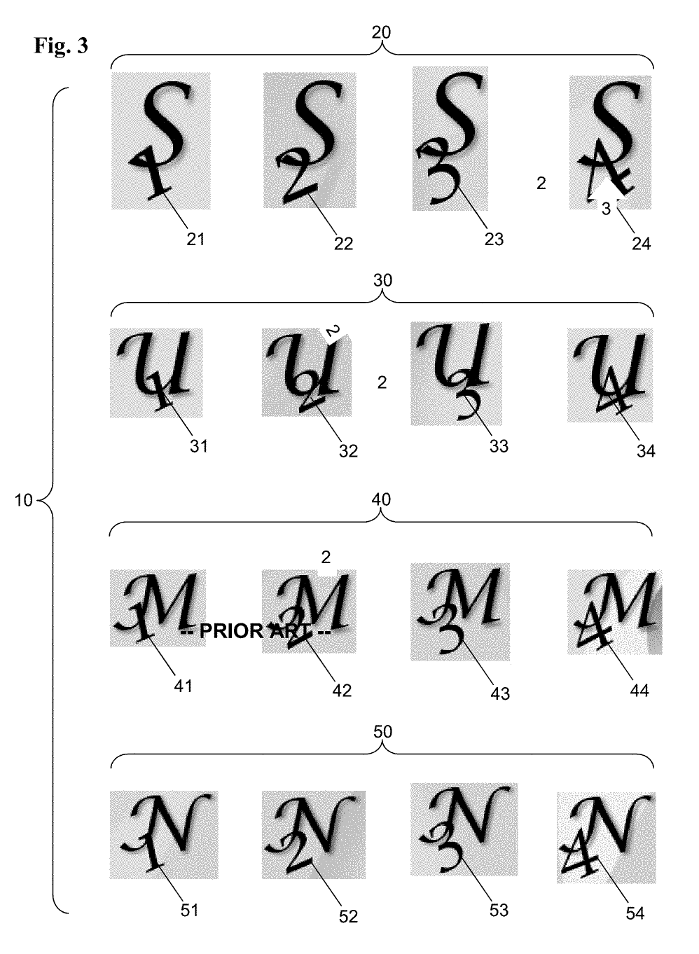

In FIG. 2, an image of the tissue of a patient is shown. In this image, a plurality of prior art markers 2, 3 are shown. In this example, four cylindrical prior art markers 2 are shown, along with one double-looped prior art marker 3. As one might imagine, reliable differentiation of the cylindrical prior art markers 2 is nearly impossible. Aspects of improved markers are introduced in FIG. 3.

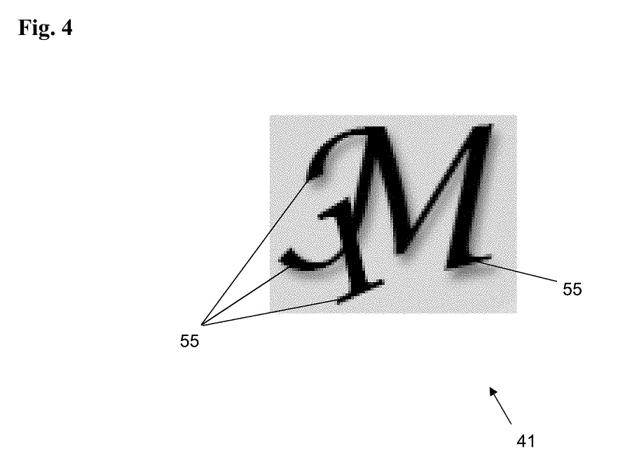

In FIG. 3, embodiments of markers 10 are shown in four series. The first series of markers 20 includes stereotactic guided biopsy sequential markers 21, 22, 23 and 24. The second series of markers 30 includes ultrasound guided biopsy sequential markers 31, 32, 33 and 34. The third series of markers 40 includes magnetic resonance imaging guided biopsy sequential markers 41, 42, 43 and 44. The fourth series of markers 50 includes lymph node biopsy sequential markers 51, 52, 53 and 54. In the embodiments depicted, each of the sequential markers includes retention features. Refer to FIG. 4 where one of the sequential markers is shown in greater detail.

In FIG. 4, a selected sequential marker 41 is shown with the retention features 55. In this illustration, the retention features 55 include irregularly shaped portions that assist in the immobilization of the sequential marker 41 once implanted. Each of the retention features 55 may be associated with a font, as is the case in this illustration, or may be provided as an additional portion of the sequential marker. The retention features 55 may include, for example, a hook, a barb, a protuberance, a point, a jagged edge, a roughened surface, a porous surface and/or any other type of surface deemed appropriate for assisting with limiting the mobility of the marker.

Generally, the markers 10 disclosed herein are fabricated from biocompatible materials. Examples of biocompatible materials include metals such as stainless steel, cobalt alloys, titanium alloys and others; ceramic materials such as aluminum oxide, zirconia, calcium phosphates, a polymeric material and others. In some embodiments, the markers are fabricated from shape-memory materials. As discussed herein, “shape-memory material” generally refers to materials that may be deformed under a physical force and assume a particular shape when the force is released and the material relaxes. Also as discussed herein, “biocompatible material” generally refers to material that will not harm, or will not substantially harm living tissue into which the material may be disposed.

Each marker may include contrast enhancing agents. For example, the markers may contain radioactive, magnetic, or sound emitting tracers that are visible to appropriate detection systems. One example of magnetic marking technology is the SENTIMAG® and MAGSEED® technology available from Mammatome of Cincinnati, Ohio. This technology makes use of markers with magnetic properties and provides a detection system. One example of a sonic technology is the SCOUT® technology available from Cianna Medical of Aliso Viejo, Calif. This technology makes use of a radar localization system that detects reflective markers. In some embodiments, the markers may be imbedded with or associated with hydrophilic material to improve ultrasound visualization.

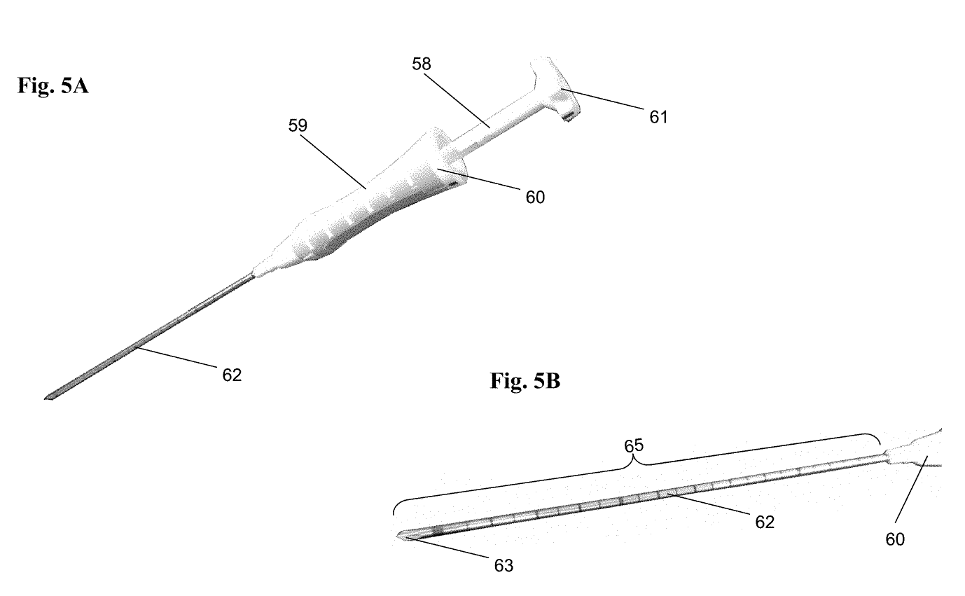

Each marker 10 may be delivered to a biopsy site by one of a variety of ways. For example, a practitioner may manually place the marker 10 at the biopsy site. In some embodiments, the marker 10 is delivered to the biopsy site by use of a introducing device, such as the one shown in FIG. 5.

In FIGS. 5A and 5B, collectively referred to herein as FIG. 5, an example of an instrument suited for delivery and implantation of the marker 10 is shown. In this example, the instrument is a trocar 60. The trocar 60 includes a body 59 to which a cannula 62 having a length 65 is attached. The length 65 may include a series of linear marking to aid the practitioner in estimating a depth within tissue of the patient. A plunger 58 is mounted within the body 59. The marker 10 is disposed at an opposing end of the plunger 58 and within the cannula 62.

When a thumb pad 61 for the plunger 58 is depressed, the plunger plunges the marker 10 through the cannula 62 and out from the distal end 63 thereof. Once the marker 10 has been pushed from the cannula 62, the retention features 50 generally aid in maintaining a position of the marker 10 within the tissue.

In some embodiments, the trocar 60 is labeled. For example, the thumb pad 61 may be adorned with an image of the particular embodiment of marker 10 with which the trocar 60 has been loaded. That is, an image of the marker 10 that is contained within the trocar 60 is disposed on the thumb pad 61. Examples of images are provided in FIG. 3.

Collectively, the delivery device (such as the trocar 60) along with the marker 10 provide a system for marking a biopsy site. The instrument may be pre-loaded with the marker 10 or be loaded with the marker 10 during the surgical procedure.

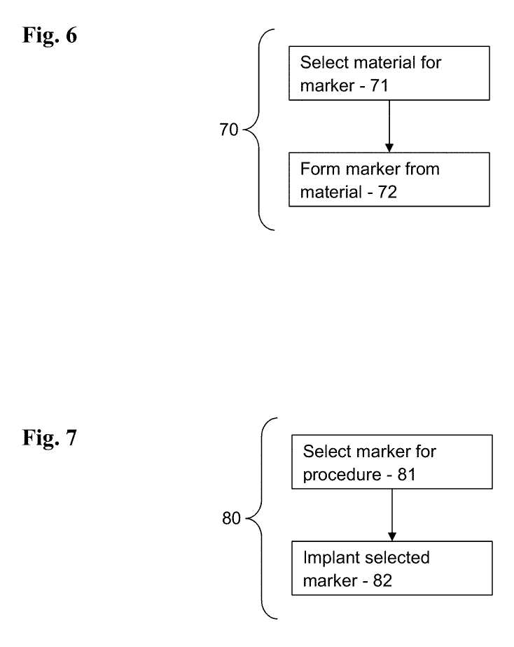

Turning to FIG. 6, a flow chart depicting an exemplary procedure for fabrication of markers 70 is shown. In a first step 71, material for the markers to be fabricated is selected. In a second step 72, the selected material is used to form the desired quantity of markers. Forming of the markers may include any one or more of a variety of processes. Examples include, without limitation, molding, machining, casting, assembling, melting, solidifying, polishing and the like. Subsequent to forming of the markers, finishing including packaging and sterilization may take place.

Turning to FIG. 7, a flow chart depicting an exemplary procedure for implanting of markers 80 is shown. In a first step 81, an appropriate marker for a given procedure is selected. Selection may involve, for example, correlating the selection process with the type of procedure and sequence of procedure undertaken. In a second step 72, the selected marker is implanted into the patient at the biopsy site. Implanting the marker may include, for example, use of the trocar or other medical interventional tools.

Having thus introduced aspects of the invention, some additional features, embodiments and considerations are now set forth.

Although disclosed herein as useful for breast and lymph node biopsies, the markers may be used in any type of interventional procedure where deemed appropriate. Among other things, the use of the markers is not limited to marking of biopsy sites. Accordingly, the use of the term “biopsy” in relation to the markers is not intended to be limiting, but merely illustrative of a medical procedure that may benefit from the technology disclosed herein.

In some embodiments, the alphanumeric characters chosen for the encoded medical information are reflective of geographical, racial or other preferences. For example, suitable coding systems in the United States may be based on Arabic numbers and the English alphabet. In some other embodiments, other characters may be used. This may be appropriate for Asian, Eastern Europe or other cultures where the patient may reside.

The markers may be used in conjunction with other techniques for marking. For example, the markers may be treated with materials, such as antibiotics to limit infection in-situ (i.e., substantially within the original place where the marker is to be disposed).

Standards for performance, materials, assembly or other such parameters are to be judged by a designer, manufacturer, user, owner, operator or other similarly interested party. No particular requirements for any standards are implied or to be inferred by the disclosure provided.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The usage of the term “exemplary” is to be construed as meaning one of many possible embodiments. The term “exemplary” is not to be construed as being necessarily indicative of a superior or superlative embodiment, although, in some instances this may be the case.

While the invention has been described with reference to exemplary embodiments, it will be understood by those skilled in the art that various changes may be made and equivalents may be substituted for elements thereof without departing from the scope of the invention. In addition, many modifications will be appreciated by those skilled in the art to adapt a particular instrument, situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A surgical marker comprising:
a biocompatible material encoded with information of medical significance, the surgical marker exhibiting a geometry suited for being disposed at a location of interest within a patient and remaining in-situ to signify the information.

2. The surgical marker as in claim 1, wherein a shape of the surgical marker comprises at least one alphanumeric character.

3. The surgical marker as in claim 2, wherein the at least one alphanumeric character is derived from at least one of the English alphabet and Arabic numbers.

4. The surgical marker as in claim 1, wherein the encoded information indicates at least one of a surgical modality and a sequence within a series of surgeries.

5. The surgical marker as in claim 1, wherein the surgical marker comprises at least one retention feature.

6. The surgical marker as in claim 5, wherein at least one retention feature comprises a hook, a barb, a protuberance, a point, a jagged edge, a roughened surface, a porous surface and/or any other type of surface deemed appropriate for assisting with limiting the mobility of the marker.

7. The surgical marker as in claim 1, wherein the biocompatible material comprises at least one of: metal, stainless steel, a cobalt alloy, a titanium alloy; a ceramic material, aluminum oxide, zirconia, calcium phosphate and a polymeric material.

8. The surgical marker as in claim 1, wherein the biocompatible material comprises shape-memory material.

9. The surgical marker as in claim 1, configured for delivery to the location of interest by use of a cannula.

10. The surgical marker as in claim 1, further comprising at least one contrast enhancing agent incorporated therewith.

11. The surgical marker as in claim 1, further comprising at least one antibiotic agent incorporated therewith.

12. A method for marking a biopsy site with a surgical marker, the method comprising:
selecting a surgical marker comprising a biocompatible material encoded with information of medical significance, the surgical marker exhibiting a geometry suited for being disposed at a location of interest within a patient and remaining in-situ to signify the information; and,
implanting the surgical marker at a surgical site.

13. The method as in claim 10, wherein the selecting comprises choosing the surgical marker according to the encoded information in correlation with at least one of a modality and a sequence in a series of surgeries.

14. The method as in claim 10, wherein the selecting further comprises one of obtaining an instrument with the surgical marker loaded therein and loading an instrument with the surgical marker.

15. The method as in claim 10, wherein the implanting comprises one of manually disposing the surgical marker at the surgical site and operating an instrument for disposing the surgical marker at the surgical site.

16. A method for fabrication of a surgical marker for marking a surgical site, the method comprising:
selecting at least one biocompatible material for forming the surgical marker; and,
forming the surgical marker into a geometry suited for being disposed at a location of interest within a patient and remaining in-situ to signify encoded information of medical significance.

17. The method as in claim 16, further comprising at least one of molding, machining, casting, assembling, melting, solidifying, polishing the surgical marker.

18. The method as in claim 16, further comprising at least one of packaging and sterilizing the surgical marker.

19. The method as in claim 16, further comprising selecting the biocompatible material from at least one of metal, stainless steel, a cobalt alloy, a titanium alloy; a ceramic material, aluminum oxide, zirconia, calcium phosphate, a polymeric material and shape-memory material.
